(19) United States
(12) Patent Application Publication
(10) Pub. No.: US 2008/0167591 A1
(43) Pub. Date: Jul. 10, 2008

(54) BACK SUPPORT

(76) Inventor: Ahmed Yahya, Southwater (GB)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 11/621,670

(22) Filed: Jan. 10, 2007

(51) Int. Cl. A61F 5/00

(52) U.S. Cl. 602/19

(57) ABSTRACT

A back support, in particular a spine supporting device, for supporting the spine of a user, the back support comprising upper and lower support members which are in use fixed to upper and lower regions of the body of the user, and a mechanical spine which interconnects the upper and lower support members, such as to transmit a load from the upper support member to the lower support member, with the lower support member being anchored to the user such as to bear the transmitted load, and thereby relieve the spine of the user of that load.

### FIELD OF THE INVENTION

The present invention relates to a back support for people with spinal conditions, and in particular lower back disc problems.

### BACKGROUND OF THE INVENTION

Frequently, people suffer with pain in their hips, buttocks, back, legs or feet as a consequence of spinal conditions, and the present invention has application to people who suffer any of these symptoms and also those whom are unfortunate enough to suffer with all these symptoms, which is commonly known as sciatica.

The present invention aims to provide an artificial support which allows the body of the wearer to remain held and supported whilst in an upright position, such as standing, sitting, walking, etc. This support alleviates the pressure on the damaged disc or discs, hence reducing the pain experienced by the individual.

The present invention has particular application for those who cannot undergo surgery or have already undergone failed surgery. The present invention also provides a non-invasive option which would suit those that do not wish to take the risk associated with a major surgical option.

Currently, the only devices available for sufferers of sciatica are rigid traction-type arrangements, which are designed to restrict the movement of the body of the user in a specific manner and are primarily intended for post-operative recovery.

The present invention, in contrast, provides for a full range of movement, which should allow the wearer near normal function. Coupled with shock-absorbing characteristics, the aim of the present invention is closely to mimic the natural actions of the discs in the spine.

There are also a large number of back braces which have a stiffened portion located to the rear of the spine, and are anchored just under the ribs of the individual to provide support, typically by a hook and loop type fastening arrangement. The present invention is quite different to such back braces in not utilizing any stiffened inserts or plates which are anchored under the ribs to the waist, but instead the upper support is implemented through the mechanical spine. Furthermore, such conventional back braces can be problematic in causing further pressure on the damaged region of the spine, so making the everyday use of the back braces impractical or too restricting.

GB-A-2159058 discloses another example of a spinal support, but this device differs from that of the present invention in that the device utilizes a supporting pad which in effect acts as a prop to the spine of the user, and does not act as a mechanical spine which transmits a substantially vertical load from an upper support member which is fixed to an upper region of the body of the user to a lower support member which is fixed to a lower region of the body of the user, here at hips and waist of the user.

There are also very many spine protectors, such as disclosed in GB-A-2225708, U.S. Pat. No. 4,680,812, U.S. Pat. No. 5,328,447 and U.S. Pat. No. 5,586,561, but these devices again do not provide a mechanical spine which transmits a substantially vertical load from an upper support member which is fixed to an upper region of the body of the user to a lower support member which is fixed to a lower region of the body user.

### SUMMARY OF THE INVENTION

In one aspect the present invention provides a back support, in particular a spine supporting device, for supporting the spine of a user, the back support comprising upper and lower support members which are in use fixed to upper and lower regions of the body of the user, and a mechanical spine which interconnects the upper and lower support members, such as to transmit a load from the upper support member to the lower support member, with the lower support member being anchored to the user such as to bear the transmitted load, and thereby relieve the spine of the user of that load.

In one embodiment the mechanical spine has shock-absorbing characteristics and provides the user with a substantially full range of movement.

In one embodiment the upper support member comprises an upper attachment element, which is located to the rear of the body of the user, to which the upper end of the mechanical spine is connected, and an upper supporting framework which is connected to the upper attachment element and fitted to the upper region of the body of the user.

In one embodiment the upper supporting framework is coupled at the front by a belt and buckle, and preferably the belt is elasticated.

In one embodiment the upper supporting framework comprises a pair of supporting shoulder straps.

In one embodiment the lower support member comprises a lower attachment element, which is located to the rear of the body of the user, to which the lower end of the mechanical spine is connected, and a lower supporting framework which is connected to the lower attachment element and fitted to the lower region of the body of the user such as to be in frictional engagement therewith.

In one embodiment the lower attachment element is coupled at the front by a belt and buckle, and the belt is preferably non-elasticated.

In one embodiment the lower supporting framework comprises a waistband, which is configured frictionally to engage the lower region of the body of the user, such as to fix the lower supporting framework to the body of the user and thereby support the load as transmitted through the mechanical spine from the upper support member.

In one embodiment the frictional engagement is sufficient to fix the position of the lower support member when supporting the load as transmitted by the mechanical spine.

In one embodiment the lower attachment element includes at least one guide aperture, and the mechanical spine includes at least one guide member which is received in the at least one guide aperture, such as to allow for longitudinal movement of the mechanical spine, either by compression or expansion.

In one embodiment the mechanical spine includes an adjustable coupling for allowing adjustment of the length of the mechanical spine in accommodating users of different size.

In one embodiment the upper support member comprises an upper attachment element, which is located to the rear of the body of the user, to which the upper end of the mechanical spine is connected, the lower support member comprises a lower attachment element, which is located to the rear of the body of the user, to which the lower end of the mechanical spine is connected, and one of the attachment elements of the upper and lower support members, preferably the lower attachment element, and one end of the mechanical spine, preferably the lower end, each comprise counterpart threaded couplings, which allow for adjustment of the length of the mechanical spine in accommodating users of different size.

In one embodiment the mechanical spine comprises a plurality of interconnected, load-bearing segments which extend between the upper and lower support members, such as to transmit the load as supported by the upper support member, and are movable relative to one another, such as to allow for movement, preferably substantially full movement, of the body of the user.

In one embodiment adjacent ones of the load-bearing segments are disposed in overlapping relation.

In one embodiment the load-bearing segments increase in size from the upper to the lower end of the mechanical spine.

In one embodiment the load-bearing segments are substantially rectangular plates which have an increasing lateral dimension.

In one embodiment the uppermost load-bearing segment is fixed to the upper support member, and the lowermost load-bearing segment and the lower support member each include inter-engageable threaded couplings, which provide for the adjustable coupling of the mechanical spine to the lower support member.

In one embodiment the mechanical spine includes a plurality of resilient elements, which are each disposed between respective ones of the load-bearing segments.

In one embodiment the resilient elements comprise rubber strips, the ends of which are adhered to respective ones of the adjacent load-bearing segments.

In one embodiment each of the resilient elements is attached at one end to the lower end of one face of one, upper load-bearing segment, passes over the lower leading edge of that load-bearing segment, between the other face of that load-bearing segment and one face of the adjacent, lower load-bearing segment, over the upper leading edge of that lower load-bearing segment, and attached at the other end to the upper end of the other face of the lower load-bearing segment.

In one embodiment the load-bearing segments each include a roller at at least one leading edge thereof, preferably extending along the length of the leading edge, such as to facilitate movement of the load-bearing segments relative to one another.

In one embodiment the mechanical spine further comprises guard plates which encompass the front and rear surfaces of the load-bearing segments and act to maintain the load-bearing segments in a substantially vertical orient.

In one embodiment the lower support member includes guide apertures, and the guard plates are fixed at the upper end thereof to the upper support member and slideably disposed in the guide apertures, such as to allow for longitudinal movement of the spine, either by compression or expansion.

In one embodiment the back support further comprises a garment cover which encloses at least the upper and lower support members.

In one embodiment the garment cover is formed of a rubber material, preferably a soft rubber material, which promotes frictional engagement of the lower support member to the lower region of the body of the user.

In another aspect the present invention provides a method of supporting the back, in particular the spine, of a user, the method comprising the steps of: fixing an upper support member to an upper region of the body of the user; fixing a lower support member to a lower region of the body of the user, wherein the lower support member is anchored to the user such as to bear a downwardly-directed load; and interconnecting a mechanical spine to the upper and lower support members, such as to transmit a load from the upper support member to the lower support member, whereby the lower support member bears the transmitted load, and thereby relieves the spine of the user of that load.

### BRIEF DESCRIPTION OF THE INVENTION

A preferred embodiment of the present invention will now be described hereinbelow by way of example only with reference to the accompanying drawings, in which:

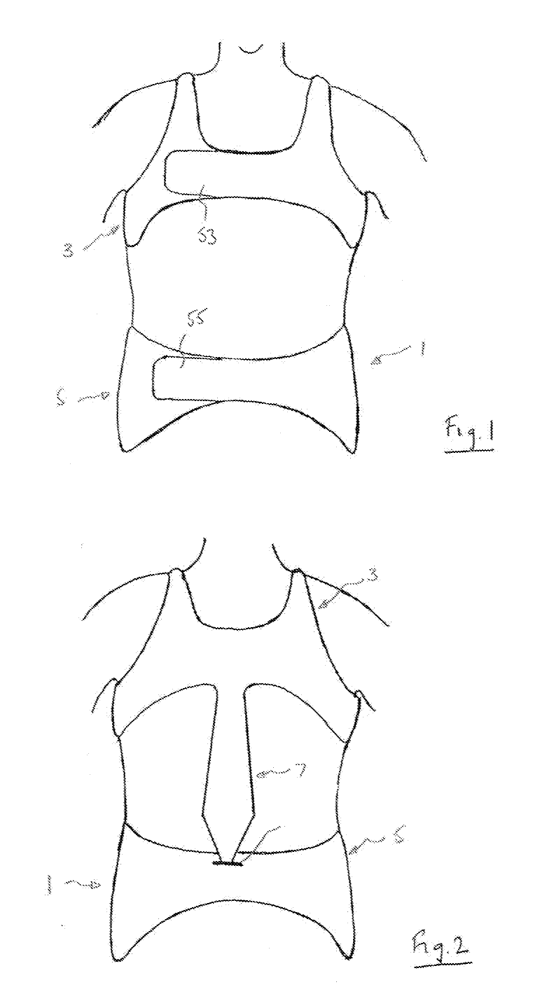

FIG. 1 illustrates a front elevational view of a spine supporting device in accordance with a preferred embodiment of the present invention, as worn by a user;

FIG. 2 illustrates a rear elevational view of the spine supporting device of FIG. 1, as worn by a user;

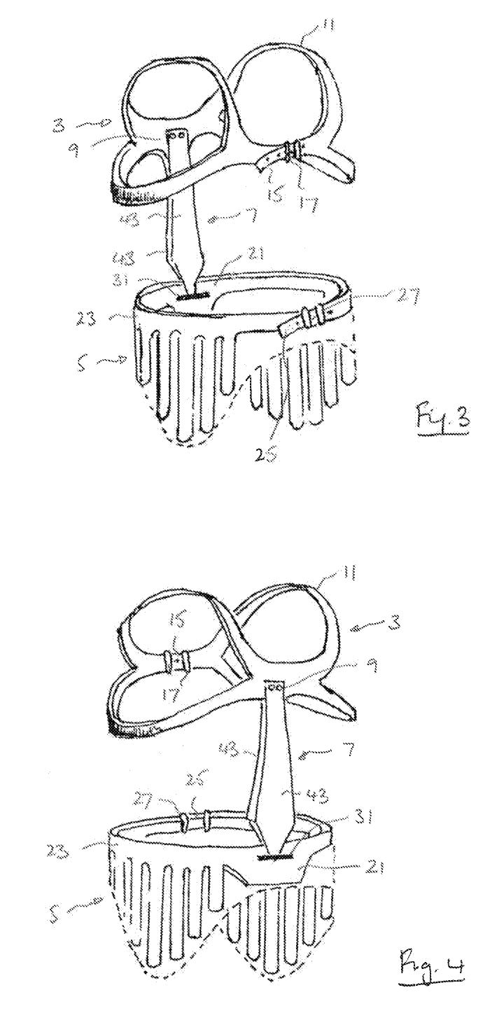

FIG. 3 illustrates a front perspective view of the spine supporting device of FIG. 1, with the covering garment removed;

FIG. 4 illustrates a rear perspective view of the spine supporting device of FIG. 1, with the covering garment removed;

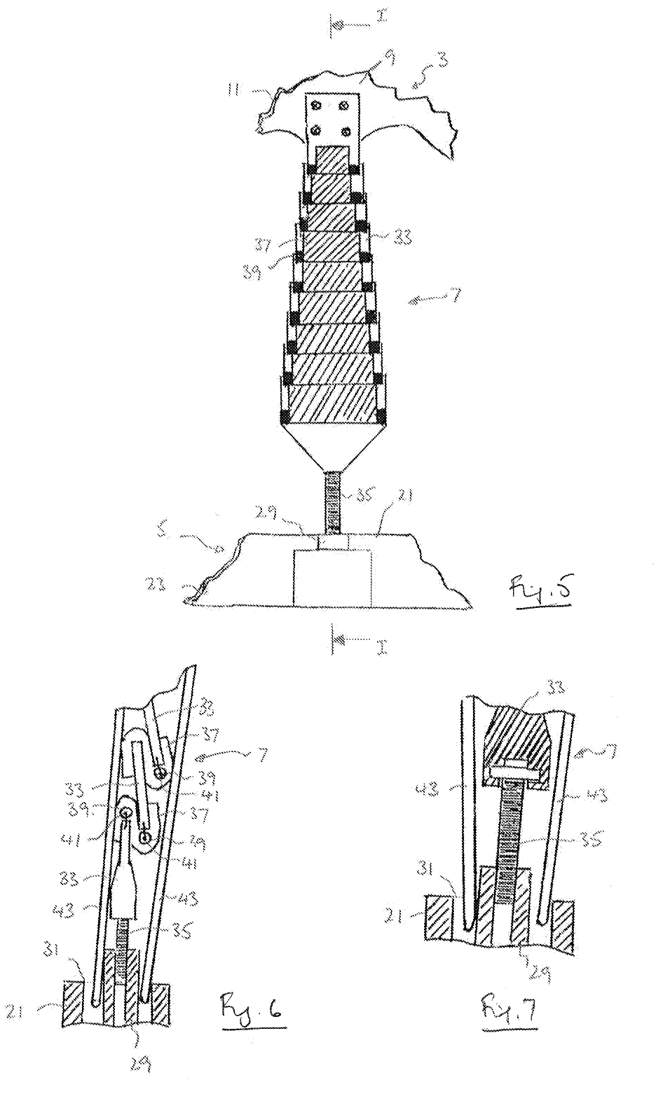

FIG. 5 shows a front elevational view of the mechanical spine of the spine supporting device of FIG. 1, with the guard plates removed;

FIG. 6 illustrates a fragmentary vertical sectional view (along section I-I in FIG. 5) of the mechanical spine of the spine supporting device of FIG. 1; and

FIG. 7 illustrates an exploded fragmentary view of the vertical sectional view of FIG. 6.

### DETAILED DESCRIPTION OF A PREFERRED EMBODIMENT

The supporting device 1 comprises upper and lower support members 3, 5, which are anchored to the user, and a mechanical spine 7, which interconnects the upper and lower support members 3, 5, such that the mechanical spine 7 transmits the load from the upper support member 5 to the lower support member 5, which is anchored such as to bear the transmitted load, and thereby relieve the spine of the user of that load. In this embodiment the function of the mechanical spine 7 is to mimic the action of a natural spine, having shock-absorbing characteristics and providing the user with a full range of movement.

In this embodiment the upper support member 3 comprises an attachment element 9, which is located to the rear of the body of the user, here at a location intermediate the shoulder blades, to which the upper end of the mechanical spine 7 is connected, and a supporting framework 11 which is connected to the attachment element 9 and coupled at the front by a belt and buckle 15, 17. In this embodiment the belt 15 is elasticated, such as to provide for the close fitting of the upper support member 3 to the body of the wearer and yet allow for the normal expansion of the chest of the user.

In this embodiment the supporting framework 11 comprises a pair of shoulder straps. In one embodiment the shoulder straps can be rigid or semi-rigid elements.

In this embodiment the lower support member 5 comprises an attachment element 21, which is located to the rear of the body of the user, here at a location intermediate the hips, to which the lower end of the mechanical spine 7 is connected, and a supporting framework 23 which is connected to the attachment element 21 and coupled at the front by a belt and buckle 25, 27. In this embodiment the belt 25 is non-elasticated.

In this embodiment, as illustrated in FIGS. 5 to 7, the attachment element 21 comprises a threaded coupling 29, here a threaded bore, to which a counterpart threaded coupling 35 of the mechanical spine 7 is coupled, in order to allow for adjustment of the length of the mechanical spine 7 in accommodating users of different size, and at least one aperture 31, here a pair of slots, through which extend guard plates 43 of the mechanical spine 7, as will be described in more detail hereinbelow.

In this embodiment the supporting framework 23 comprises a waistband, which is configured frictionally to engage the body of the user, such as to fix the supporting framework 23 to the body of the user and thereby support the load as transmitted through the mechanical spine 7 from the upper support member 3. In the present invention, a sufficient frictional engagement is necessary so as to fix the position of the lower support member 5 when supporting the load as transmitted by the mechanical spine 7.

The mechanical spine 7 comprises a plurality of interconnected, load-bearing segments 33 which extend between the attachment elements 9, 21 of the upper and lower support members 3, 5, such as to transmit the load as supported by the upper support member 3, and are movable relative to one another, such as to allow for full movement of the body of the user.

In this embodiment adjacent ones of the load-bearing segments 33 are disposed in overlapping relation.

In this embodiment the load-bearing segments 33 increase in size from the upper to the lower end of the mechanical spine 7.

In one embodiment the load-bearing segments 33 are substantially rectangular plates which have an increasing lateral dimension.

In one embodiment the load-bearing plates are formed of a plastics material.

In this embodiment the uppermost load-bearing segment 33 is fixed to the attachment element 9 of the upper support member 3, and the lowermost load-bearing segment 33 includes a threaded coupling 35, here a threaded screw, which is coupled to the counterpart threaded coupling 29 at the attachment member 21 of the lower support member 5, such as to provide for the adjustable coupling of the mechanical spine 7 to the lower support member 5.

In this embodiment the threaded coupling 35 is rotatably coupled to the lowermost load-bearing segment 33, such as to allow for adjustment of the length of the mechanical spine 7 without requiring rotation of the mechanical spine 7 relative to the lower support member 5. In this embodiment the threaded coupling 35 is rotated by engagement with an engagement feature on the lower end of the threaded coupling 35.

In this embodiment the mechanical spine 7 includes a plurality of resilient elements 37, which are each disposed between respective ones of the load-bearing segments 33.

In this embodiment the resilient elements 37 comprise rubber strips, the ends of which are adhered to respective ones of the adjacent load-bearing segments 33.

In this embodiment each of the resilient elements 37 is attached at one end to the lower end of one, the rear, face of one, upper load-bearing segment 33, passes over the lower leading edge of that load-bearing segment 33, between the other, front face of that load-bearing segment 33 and one, the rear, face of the adjacent, lower load-bearing segment 33, over the upper leading edge of that lower load-bearing segment 33, and attached at the other end to the upper end of the other, front face of the lower load-bearing segment 33.

In another embodiment the resilient elements 37 could comprise a contained fluid.

In this embodiment the load-bearing segments 33 each include a roller 39 at at least one leading edge thereof, here extending along the length of the leading edge, such as to facilitate movement of the load-bearing segments 33 relative to one another, here through engagement with the respective resilient elements 37.

In this embodiment the roller 39 is supported about its axis by a connecting pin 41.

In this embodiment the roller 39 is formed of stainless steel, but could be formed of other similar materials.

In this embodiment the mechanical spine 7 further comprises guard plates 43 which encompass the front and rear surfaces of the load-bearing segments 33. The guard plates 43 serve the functions of maintaining the load-bearing elements 33 in a generally-vertical orient and also prevent the trapping of the skin of the user or the clothing.

In this embodiment the guard plates 43 are fixed at the upper end thereof to the attachment element 9 of the upper support member 3 and are slideably disposed in the aperture 31, here the respective slots in the attachment element 9. With this configuration, longitudinal movement of the load-bearing segments 33, either by way of compression or extension, can be accommodated.

In this embodiment the guard plates 43 are provided by a stiffened plastic framework.

The spine supporting device 1 further comprises a garment cover 51 which encloses the upper and lower support members 3, 5 and the mechanical spine 7.

In this embodiment the garment cover 51 is formed of a soft rubber material, here in the form of inner and outer skins, which are stitched and glued to the upper and lower support members 3, 5 and the guard plates 43 of the mechanical spine 7. Such a rubber material assists in fixing the lower support member 5 to the hips and waist of the user by means of promoting frictional engagement with the skin of the user.

In this embodiment the outer skin of the garment cover 51 includes hook and loop type fasteners 53, 55 across the chest and waist, such as to provide for releasable fitting of the garment cover 51.

Finally, it will be understood that the present invention has been described in its preferred embodiment and can be modified in many different ways without departing from the scope of the invention as defined by the appended claims.

For example, numerous configurations in the construction of the mechanical spine 7 and the materials thereof could be used to provide the desired shock-absorbing characteristics and full scope of movement required by the user.

Also, the described embodiment has been adapted for a male user, and it will be understood that the present invention can be readily adapted for female users, which would merely require re-configuration of the upper support member 3.

1. A back support, in particular a spine supporting device, for supporting the spine of a user, the back support comprising upper and lower support members which are in use fixed to upper and lower regions of the body of the user, and a mechanical spine which interconnects the upper and lower support members, such as to transmit a load from the upper support member to the lower support member, with the lower support member being anchored to the user such as to bear the transmitted load, and thereby relieve the spine of the user of that load.

2. The back support of claim 1, wherein the mechanical spine has shock-absorbing characteristics and provides the user with a substantially full range of movement.

3. The back support of claim 1, wherein the upper support member comprises an upper attachment element, which is located to the rear of the body of the user, to which the upper end of the mechanical spine is connected, and an upper supporting framework which is connected to the upper attachment element and fitted to the upper region of the body of the user.

4. The back support of claim 3, wherein the upper supporting framework is coupled at the front by a belt and buckle, and preferably the belt is elasticated.

5. The back support of claim 3, wherein the upper supporting framework comprises a pair of supporting shoulder straps.

6. The back support of claim 1, wherein the lower support member comprises a lower attachment element, which is located to the rear of the body of the user, to which the lower end of the mechanical spine is connected, and a lower supporting framework which is connected to the lower attachment element and fitted to the lower region of the body of the user such as to be in frictional engagement therewith.

7. The back support of claim 6, wherein the lower attachment element is coupled at the front by a belt and buckle, and the belt is preferably non-elasticated.

8. The back support of claim 6, wherein the lower supporting framework comprises a waistband, which is configured frictionally to engage the lower region of the body of the user, such as to fix the lower supporting framework to the body of the user and thereby support the load as transmitted through the mechanical spine from the upper support member.

9. The back support of claim 8, wherein the frictional engagement is sufficient to fix the position of the lower support member when supporting the load as transmitted by the mechanical spine.

10. The back support of claim 6, wherein the lower attachment element includes at least one guide aperture, and the mechanical spine includes at least one guide member which is received in the at least one guide aperture, such as to allow for longitudinal movement of the mechanical spine, either by compression or expansion.

11. The back support of claim 1, wherein the mechanical spine includes an adjustable coupling for allowing adjustment of the length of the mechanical spine in accommodating users of different size.

12. The back support of claim 1, wherein the upper support member comprises an upper attachment element, which is located to the rear of the body of the user, to which the upper end of the mechanical spine is connected, the lower support member comprises a lower attachment element, which is located to the rear of the body of the user, to which the lower end of the mechanical spine is connected, and one of the attachment elements of the upper and lower support members, preferably the lower attachment element, and one end of the mechanical spine, preferably the lower end, each comprise counterpart threaded couplings, which allow for adjustment of the length of the mechanical spine in accommodating users of different size.

13. The back support of claim 1, wherein the mechanical spine comprises a plurality of interconnected, load-bearing segments which extend between the upper and lower support members, such as to transmit the load as supported by the upper support member, and are movable relative to one another, such as to allow for movement, preferably substantially full movement, of the body of the user.

14. The back support of claim 13, wherein adjacent ones of the load-bearing segments are disposed in overlapping relation.

15. The back support of claim 13, wherein the load-bearing segments increase in size from the upper to the lower end of the mechanical spine.

16. The back support of claim 15, wherein the load-bearing segments are substantially rectangular plates which have an increasing lateral dimension.

17. The back support of claim 13, wherein the uppermost load-bearing segment is fixed to the upper support member, and the lowermost load-bearing segment and the lower support member each include inter-engageable threaded couplings, which provide for the adjustable coupling of the mechanical spine to the lower support member.

18. The back support of claim 13, wherein the mechanical spine includes a plurality of resilient elements, which are each disposed between respective ones of the load-bearing segments.

19. The back support of claim 18, wherein the resilient elements comprise rubber strips, the ends of which are adhered to respective ones of the adjacent load-bearing segments.

20. The back support of claim 19, wherein each of the resilient elements is attached at one end to the lower end of one, the rear, face of one, upper load-bearing segment, passes over the lower leading edge of that load-bearing segment, between the other, front face of that load-bearing segment and one, the rear, face of the adjacent, lower load-bearing segment, over the upper leading edge of that lower load-bearing segment, and attached at the other end to the upper end of the other, front face of the lower load-bearing segment.

21. The back support of claim 13, wherein the load-bearing segments each include a roller at at least one leading edge thereof, preferably extending along the length of the leading edge, such as to facilitate movement of the load-bearing segments relative to one another.

22. The back support of claim 13, wherein the mechanical spine further comprises guard plates which encompass the front and rear surfaces of the load-bearing segments and act to maintain the load-bearing segments in a substantially vertical orient.

23. The back support of claim 22, wherein the lower support member includes guide apertures, and the guard plates are fixed at the upper end thereof to the upper support member and slideably disposed in the guide apertures, such as to allow for longitudinal movement of the spine, either by compression or expansion.

24. The back support of claim 1, further comprising a garment cover which encloses at least the upper and lower support members.

25. The back support of claim 24, wherein the garment cover is formed of a rubber material, preferably a soft rubber material, which promotes frictional engagement of the lower support member to the lower region of the body of the user.

26. A method of supporting the back, in particular the spine, of a user, the method comprising the steps of:
fixing an upper support member to an upper region of the body of the user;
fixing a lower support member to a lower region of the body of the user, wherein the lower support member is anchored to the user such as to bear a downwardly-directed load; and
interconnecting a mechanical spine to the upper and lower support members, such as to transmit a load from the upper support member to the lower support member, whereby the lower support member bears the transmitted load, and thereby relieves the spine of the user of that load.

27. A back support, which is comprised of upper and lower portions, which are interconnected via a mechanical spine at the back of the individual. The upper and lower portions of this aid are made of a soft rubber material, which forms the outer skin of the garment. This assists in the securing of the garment to the individual's hips and waist by means of friction between the skin and the mentioned rubber garment. The strength of the aid is achieved through an internal framework structure, which is preferably stitched and glued into the rubber garment. The upper and lower structural framework is held to the body by means of conventional belts and buckles, the upper belt being elastic in nature. The outer rubber skin of the garment is held to the body by hook and loop type fasteners across the chest and waist.
